(19) United States
(12) Patent Application Publication
(10) Pub. No.: US 2018/0345034 A1
(43) Pub. Date: Dec. 6, 2018

(54) MYOPIA INHIBITION APPARATUS AND OCULAR METHOD

(71) Applicant: Peter Butzloff, Saint David, ME (US)

(72) Inventor: Peter Butzloff, Saint David, ME (US)

(21) Appl. No.: 15/693,369

(22) Filed: Aug. 31, 2017

Related U.S. Application Data
(60) Provisional application No. 62/516,029, filed on Jun. 6, 2017.

(51) Int. Cl. A61N 5/06

(52) U.S. Cl. CPC A61N 5/0613; A61N 5/0618; A61N 2005/0659; A61N 2005/0648; A61N 2005/0644; A61N 2005/0663

(57) ABSTRACT

Illumination apparatus, ocular apparatus, and ocular method for treating at least one eye. Illuminator illuminates eyes with 100 lux of monochromatic red light of 640 nm to 690 nm. Illuminator controls progressive myopia leading to excessive axial elongation in a juvenile or to ameliorate macular degeneration in an aging adult. Illuminator provides indirect light or diffuse light. Illuminator provides illuminance values from 2,000 lux to 30,000 lux, with a nominal indirect total combined light exposure of 9000 lux. Illuminator provides greater than 1 lux of monochromatic violet-blue light from 440 nm to 484 nm. Illuminator minimizes light wavelengths from 484 nm to 640 nm, and eliminates light having of wavelengths at or near to 550 nm. Illuminator provides visible display images and invisible illumination, with the invisible illumination being greater than 2 Watts per areal centimeter of invisible, continuous, diffuse non-graphic monochromatic Near Infrared (NIR) light directed at ocular tissues.

### CROSS-REFERENCE TO RELATED APPLICATIONS

This application claims priority to Provisional Patent Application No. 62/516,029, entitled MYOPIA INHIBITION DEVICE AND OCCULAR METHOD, filed on 6 Jun. 2017, the entire contents of which are hereby completely incorporated herein by reference.

### BACKGROUND

This invention relates to treatment ocular disorders, in general, and to treatment of myopia, in particular.

Myopia, or near-sightedness, often is associated with excessive eyeball elongation, causing a vision condition in which people can see close objects clearly, but objects farther away appear blurred. This elongation may be a result of excessive cornea curvature, eyeball elongation, or both. Myopia affects nearly 30 percent of the U.S. population, developing first in school-aged children. People with myopia may have difficulty clearly seeing a movie, TV screen, or the whiteboard in school. While myopia is thought to be an inherited condition, some believe that the progression of myopia can be influenced by childhood environments characterized by substandard indoor lighting and a lack of sufficient natural sunlight exposure, during the years of human childhood and teenage eye growth. Natural sunlight in dosages of about 30,000 LUX over a period of about 6 hours per day can be a typical dosage for juvenile human beings that are able to play or work freely in the open countryside.

With the rise of education and requirements for study, more and more children are now required to spend substantial parts of their day in classrooms under dim or substandard lighting conditions. Modern school conditions require a child to sit at a desk or a computer at indoor locations under what is often dim light. In order to save energy, ambient lighting may be reduced and light intensity decreased. In that light, children must read books, view computers and smart telephone graphic display screens, or interact with virtual reality tools. It is unfortunate that these passive or active technological tools requiring human visual attention have resulted in their use at dim locations or are designed to minimize illumination power and therefore require graphic interfaces needing environments having low ambient light levels for proper perception and operation of their intended functions.

Myopic changes to eyeball shape are not classified as a disease, being correctable by, without limitation, eyeglasses, contact lenses, or various forms of invasive surgery, such as lens replacement or Lasik surgery. However, it is known that having myopia caused by elongated eyeballs increases the risk of developing glaucoma in late adult life. Further, adults with extreme myopia can be prone to myopic macular degeneration later in life. Of long term importance, is the impact of degraded vision to the ability of human beings to see without corrective lenses or corrective surgery.

Apparently, the human body has evolved to control eyeball growth in childhood by using a feedback mechanism. Melanopsin is a type of photopigment belonging to a larger family of light-sensitive retinal proteins called opsins, and is an important optical sensor molecule that is found in the retina, with a maximum sensitivity at about 480 nm. Thus, light reaching the retina has an impact on the regulation of eye growth. Other light receptors have been implicated in regulation of eye growth. Even so, lack of understanding of these mechanisms which regulate growth of the eyes of the human child may contribute to the creation of devices and environments that tend to encourage the development of myopia in juvenile eyes.

Typically, commercial lighting devices and methods of device interfaces are based on what is aesthetically-pleasing, or what can be seen by the adult human eye. The use of computers and visual interface devices serves increasing human populations, and has no doubt served a good purpose in education, efficiency, and mental empowerment. However, the inability to reverse the impact of substandard lighting in these “state of the art” devices, and their functional characteristics, from reaching children significantly contributes to widespread myopia and vision decline. Methods of restricting time spent indoors or away from artificial devices, as well as reducing time spent under fluorescent, incandescent, and poorly designed solid state light lighting, are no longer practical or appropriate alternatives for children in many societies. No present commercial solution exists or has been suggested for the explicit purpose of utilizing the frequencies of light important to biological self-regulation of juvenile ocular growth as an incidental part of the lighting or graphic display function. Such an apparatus and method are needed.

### SUMMARY

The embodiments herein include illumination apparatus, ocular apparatus, and ocular method for treating at least one eye. Embodiments of the apparatus can include an illuminator configured to illuminate human eyes with at least 100 lux of monochromatic light having red wavelengths in the range of about 640 nm to about 690 nm, and configured to increase at least one of perfusion by blue light-initiated regulatory hormones, by ocular blood flow, or by ocular tissue oxygenation. This perfusion enhancement facilitates the transport of nocturnal circadian hormones to better reach substantially all ocular tissues. In one embodiment, the illuminator is configured to control progressive myopia leading to excessive axial elongation in a juvenile human eye. In another embodiment, the illuminator is configured to control macular degeneration in an aging adult human eye. In still another embodiment, the illuminator is configured to provide indirect light or diffuse light. In yet another embodiment, the illuminator is configured to provide illuminance values from about 2,000 lux to about 30,000 lux, with a nominal indirect total combined light exposure of about 9000 lux. In other embodiments, the illuminator includes a wearable ocular device. Embodiments of the wearable ocular device includes one of an eye mask, goggles, or a pair of glasses. In yet other embodiments, the illuminator includes a handheld device. Embodiments of the handheld device includes one of a phone, a tablet computer, or a laptop computer. In still other embodiments, the illuminator includes a stand-alone device. An embodiment of a stand-alone device includes at least one illumination panel.

In embodiments to control progressive myopia leading to excessive axial elongation in a juvenile human eye, the illuminator is configured to provide greater than about 1 lux of monochromatic light having violet-blue wavelengths in the range of about 440 nm to about 484 nm. Such an illuminator also may include embodiments configured to minimize light having wavelengths from about 484 nm to about 640 nm, and configured to substantially eliminate light having of wavelengths at or near to about 550 nm, wherein the melanopsin receptors enabling circadian cycle entrainment are stimulated at a greater amount than that of rod and cone receptors functioning to interpret environmental visual information. In yet other embodiments, the illumination apparatus has an illuminator is further configured to provide visible display images and invisible illumination, and wherein the invisible illumination comprises greater than about 2 Watts per areal centimeter of invisible, continuous, diffuse non-graphic monochromatic Near Infrared (NIR) light directed at ocular tissues. The illuminator also is configured to increase at least one of perfusion by blue light-initiated regulatory hormones, by ocular blood flow, or by ocular tissue oxygenation. This perfusion enhancement facilitates the transport of nocturnal circadian hormones to better reach substantially all ocular tissues.

Also included is embodiments of a method for illuminating a human eye, including illuminating the human eye with greater than about 100 lux of monochromatic light having red wavelengths in the range of about 640 nm to about 690 nm.

Embodiments of an ocular apparatus are provided, including an illuminator configured to illuminate human eyes with at least 100 lux of monochromatic light having red wavelengths in the range of about 640 nm to about 690 nm, wherein the illuminator is further configured to provide illuminance values from about 2,000 lux to about 30,000 lux, with a nominal indirect total combined light exposure of about 9000 lux, wherein the illuminator is further configured to provide greater than about 1 lux of monochromatic light having violet-blue wavelengths in the range of about 440 nm to about 484 nm, wherein the illuminator is further configured to minimize light having wavelengths from about 484 nm to about 640 nm, and configured to substantially eliminate light having of wavelengths at or near to about 550 nm, wherein the melanopsin receptors enabling circadian cycle entrainment are stimulated at a greater amount than that of rod and cone receptors functioning to interpret environmental visual information, wherein the illuminator is further configured to provide visible display images and invisible illumination, and wherein the invisible illumination comprises greater than about 2 Watts per areal centimeter of invisible, continuous, diffuse non-graphic monochromatic Near Infrared (NIR) light directed at ocular tissues.

Embodiments of the ocular apparatus include a wearable ocular device, a handheld device, or a stand-alone device. In some embodiments of the ocular apparatus, the illuminator is configured to control progressive myopia in a juvenile human eye. In other embodiments of the ocular apparatus, the illuminator is configured to control macular degeneration in an aging adult human eye.

Embodiments of the method include inhibiting progressive myopia in a juvenile human eye. Embodiments of the method for illuminating a human eye further include illuminating the human eye with greater than about 1 lux of monochromatic light having violet-blue wavelengths in the range of about 440 nm to about 484 nm; providing the human eye with illuminated light having illuminance values from about 2,000 lux to about 30,000 lux; and minimizing illuminated light having wavelengths from about 484 nm to about 640 nm, wherein progressive myopia leading to excessive axial elongation in a juvenile human eye is controlled. Method embodiments also may include ameliorating macular degeneration in an aging adult eye.

Method embodiments may further include illuminating the human eye with visible digital display images and invisible, diffuse irradiation emission, wherein the invisible, diffuse irradiation includes greater than about 2 Watts per areal centimeter of invisible, continuous, diffuse non-graphic monochromatic near-infrared light (NIR) having wavelengths from about 690 nm to about 950 nm, and having a spectral full width at half maximum of less than 150 nm, wherein the NIR light is directed at ocular tissues. Embodiments also include illuminating the human eye with at least about 1 Lux of ambient visible light, wherein the visible light contains blue wavelengths from about 400 nm to about 480 nm, and increasing at least one of perfusion by blue light-initiated regulatory hormones, by ocular blood flow, or by ocular tissue oxygenation. This perfusion enhancement facilitates the transport of nocturnal circadian hormones to better reach substantially all ocular tissues.

Embodiments of an ocular apparatus are provided, including an illuminator configured to illuminate human eyes with at least 100 lux of monochromatic light having red wavelengths in the range of about 640 nm to about 690 nm. The illuminator is further configured to provide illuminance values from about 2,000 lux to about 30,000 lux, with a nominal indirect total combined light exposure of about 9000 lux. The illuminator is further configured to provide greater than about 1 lux of monochromatic light having violet-blue wavelengths in the range of about 440 nm to about 484 nm. The illuminator is further configured to minimize light having wavelengths from about 484 nm to about 640 nm, and configured to substantially eliminate light having of wavelengths at or near to about 550 nm, wherein the melanopsin receptors enabling circadian cycle entrainment are stimulated at a greater amount than that of rod and cone receptors functioning to interpret environmental visual information. The illuminator is further configured to provide visible display images and invisible illumination, wherein the invisible illumination comprises greater than about 2 Watts per areal centimeter of invisible, continuous, diffuse non-graphic monochromatic Near Infrared (NIR) light directed at ocular tissues, and wherein the illuminator is further configured to increase at least one of perfusion by blue light-initiated regulatory hormones, by ocular blood flow, or by ocular tissue oxygenation.

### BRIEF DESCRIPTION OF THE DRAWINGS

The invention is generally shown by way of reference to the accompanying drawings in which:

FIG. 1 is a graph representative of natural direct circumsolar irradiance at the surface of the Earth;

FIG. 2 is a perspective illustration of a support spar, in accordance with the present teachings of the invention;

FIG. 3 is an exploded view of an embodiment of anti-myopia illuminator, in accordance with the present teachings of the invention;

FIG. 4 is an alternative form of support spars of FIG. 3, in a beveled shape, in accordance with the present teachings of the invention;

FIG. 5 is a plan view of one illuminator panel arrangement, in accordance with the present teachings of the invention;

FIG. 6 is a plan view of a two illuminator panel arrangement, in accordance with the present teachings of the invention;

FIG. 7 is a top view of the two illuminator panel arrangement of FIG. 6, in accordance with the present teachings of the invention;

FIG. 8 is a top view of a three illuminator panel arrangement including the illuminator panel arrangement of FIG. 5, in accordance with the present teachings of the invention;

FIG. 9 is an illustration of a “smart phone” handheld ocular device having supplemental anti-myopia ocular illuminating sources, in accordance with the present teachings of the invention;

FIG. 10 is an illustration of a handheld device having a Supplemental Ocular Light Dose Adapter, in accordance with the present teachings of the invention;

FIG. 11 is an illustration of a computer having a Supplemental Ocular Light Dose Adapter, in accordance with the present teachings of the invention;

FIG. 12 is an illustration of corrective eyewear having a pair of corrective lenses, in accordance with the present teachings of the invention;

FIG. 13 is an illustration of a pair of goggles, providing a head's-up display (HUD), in accordance with the present teachings of the invention;

FIG. 14 is an illustration of a soft ocular sleeping mask, in accordance with the present teachings of the invention;

FIG. 15 is an illustration of a lamp for long term delivery of treatment of ocular tissue, in accordance with the present teachings of the invention;

FIG. 16 is a block diagram of an embodiment of a therapeutic optical controller, in accordance with the present teachings of the invention; and

FIG. 17 is a block diagram depiction of methods for illuminating a human eye to achieve an ophthalmic treatment, in accordance with the present teachings of the invention.

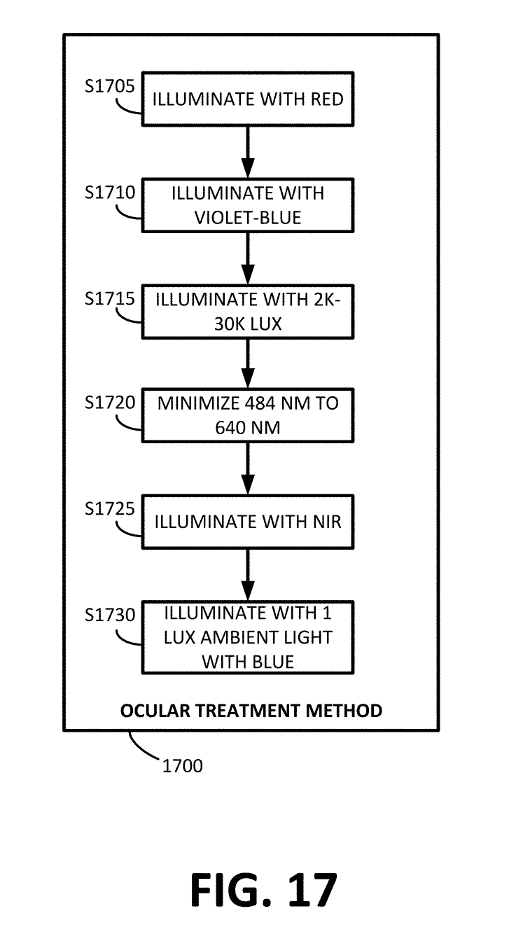

Some embodiments are described in detail with reference to the related drawings. Additional embodiments, features and/or advantages will become apparent from the ensuing description or may be learned by practicing the invention. In the figures, which are not drawn to scale, like numerals refer to like features throughout the description. The following description is not to be taken in a limiting sense, but is made merely for the purpose of describing the general principles of the invention.

### DETAILED DESCRIPTION OF THE EMBODIMENTS

Apparatus and methods are provided, which may be beneficial to individuals with myopia, in particular, children with have shown signs of, or have developed, myopia. Some adults at risk for macular degeneration also may find benefit.

The term “inhibition” as used herein means a physiological response to light exposure conditions at specific wavelengths of light and at a minimum intensity of light for a duration of about 6 hours in a typical day that is able to result in a desirable and natural long term biological response of reduced and controlled growth of ocular tissues in growing juveniles. The term, “biological activity”, as used herein, means any physiological or behavioral activity of an organism.

It is known that ocular bioregulatory mechanisms exist, which may affect the length of the eyeball. For example, melanopsin cells are intrinsically photosensitive and respond most strongly to short-wavelength light in the blue portion of the visual spectrum. They are thought to contribute to eyeball length regulation. Biological cryptochrome and phytochrome molecules collectively serve as biological signal mechanisms in blue and red wavelengths, respectively, to provide environmental growth regulation to the eyes of children. The wavelengths used by these regulatory molecules are a human genetic heritage that are also present in primitive organisms; lower animals use them as clocks or regulators of biological metabolism, and such molecules are also present in the highly evolved vision of modern higher organisms. A lack of the proper functional understanding of cryptochromes and phytochromes to the regulation of growth of the eyes of the growing human child may contribute to the creation of devices and environments that tend to encourage the development of myopia in juvenile eyes.

In is believed that the ambient natural solar light dosage of 30,000 Lux or 30,000 lumens per square meter is the typical average dosage of natural sunlight will enter into the eyeballs of a juvenile human being over a daytime period of one day of normal biological activity. This sunlight can have a blackbody radiation maximum at 550 nm. Ocular tissue growth inhibition can be achieved in part when selected portions of the solar spectrum dosage enters into the ocular tissues. One selected spectrum portion, representing violet to blue wavelengths of light, range from about 360 nm to about 460 nm tends to activate cryptochrome hormones. Regions of interest for inhibition of biological growth for cryptochrome signaling activity can be between about 400 nm to about 460 nm. Another selected spectrum portion, generally representing red to infrared wavelengths, from about 510 nm to about 900 nm, tends to activate phytochrome hormones.

Regions of interest for inhibition of biological growth for phytochrome signaling activity includes a first region of red wavelengths at about 610 nm to about 660 nm together with a second region of infrared near 860 nm. The activation of phytochromes and cryptochromes can serve interactive complementary and synergistic roles in overall growth regulation, however the long wavelength region of red to infrared can provide the primary biological growth inhibition signal in living plant and animal organisms including the human organism. The cryptochromes may regulate the circadian (day and night) cycle, so that growth is slowed according to the time of day. In addition, the phytochromes may regulate the overall growth according to the total amount of light exposure in any given day. The light that enters the eye in the violet and blue wavelengths typically focuses to a region just short of the retina; the light that enters the eye in the red and infrared regions typically focuses to a region just behind or beyond the surface of the retina. Collectively, these relatively narrow bands of wavelengths that can provide biological growth inhibition signals in ocular tissues can consist of less than 10 percent of the light irradiance, in watts per square meter, on a sunny day at noon at the surface of the Earth. Therefore, irradiation centered on these biological signaling wavelengths can be a small fraction of those wavelengths required to interpret a natural or artificial visual field produced by the graphical display of a device. Such irradiation can increase at least one of perfusion by blue light-initiated regulatory hormones, by ocular blood flow, or by ocular tissue oxygenation. This perfusion enhancement facilitates the transport of nocturnal circadian hormones to better reach substantially all ocular tissues.

Nocturnal exposure to NIR, red light at or above about 600 nm, or both, and simultaneously and substantially eliminating exposure to light below about 600 nm, can be useful to induce and to maintain beneficial blood flow to ocular tissues, which removes cryptochrome-induced growth enzymes, as well as encourages the diffusion of nocturnal inhibitory enzymes. Exposure to short frequency light, especially at blue wavelengths activates the daytime part of the circadian cycle. This entrainment is better propagated by perfusion of hormones directed into substantially all ocular tissues by increased oxygenated blood flow as a result of concurrent exposure to wavelengths of light greater than about 600 nm.

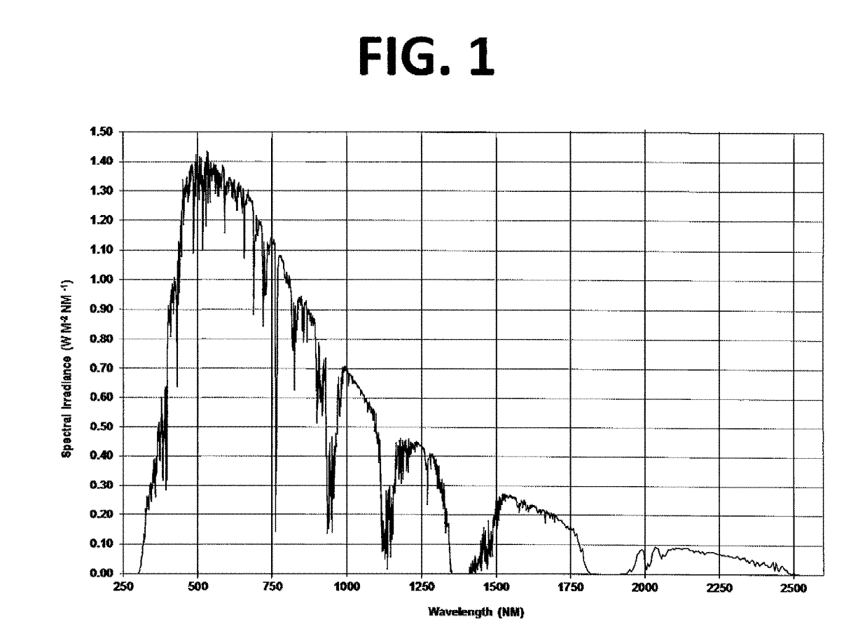

FIG. 1 is a graph obtained from the American Standard Test Method (ASTM) G173-03, Standard Tables for Reference Solar Spectral Irradiances: Direct Normal and Hemispherical on 37° Tilted Surface, which may be representative of natural direct circumsolar irradiance at the surface of the Earth, and which may be available at https://www.astm.org/Standards/G173.htm. FIG. 1 may be useful to understand the natural context of the ocular light useful for inhibition of biological growth mechanisms used by the various embodiments herein. To use this graph, multiply the wavelength in nanometers (nm) read on the x-axis by the value of the standard irradiance read by the value of the y-axis in accordance with the instructions provided by ASTM G173-03, available through ASTM International, West Conshohocken, Pa. USA. Examination of the area under the curve shown in FIG. 1 shows the narrow bands of light energy described in this specification and responsible for the signaling of biological growth regulation in the eye are collectively less than 10 percent of the sum total area of energy available for a typical solar irradiance spectrum that reaches the surface of the Earth. Therefore, it is not necessary to reproduce the relative features of this natural solar spectrum by artificial means.

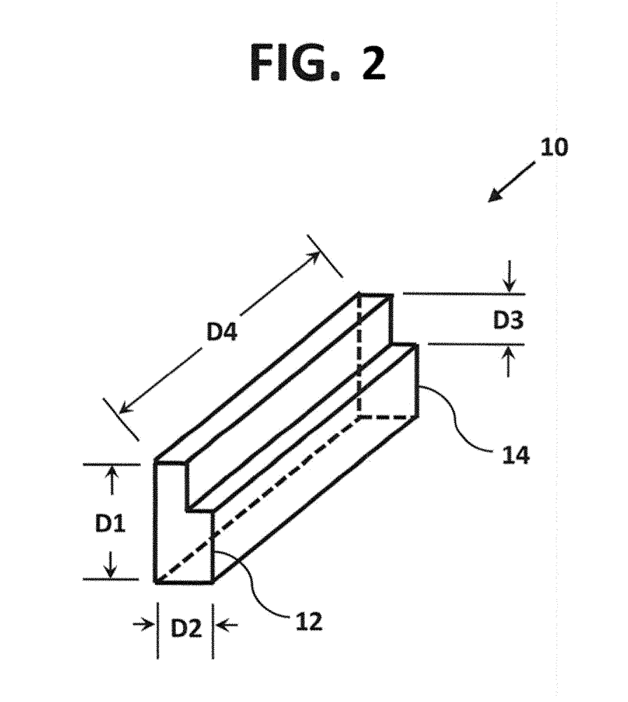

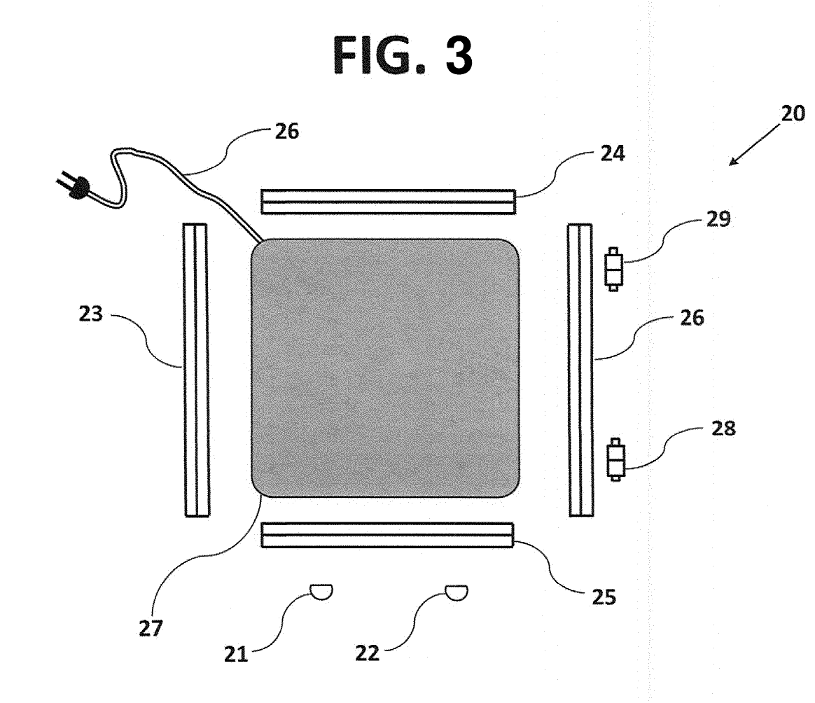

FIG. 2 is a perspective view of an embodiment of support spar 10, which may be composed of wood, plastic, metal, or another similar material which may provide a semi-rigid or a rigid structure. Such support spar 10 can be a constituent structural frame element of the devices illustrated in FIGS. 3-6. Distal ends 12 and 14 can be bounded by dimensions D1 and D2 with a chamfer of depth D3 extending along the axial length of the support spar 16. Generally, dimension D1 can be greater than either dimension D2 or dimension D3.

FIG. 3 depicts an exploded view of an embodiment of anti-myopia illuminator 20 having light source 27 configured to generate nominal blue light at about 460 nm or nominal red light at about 660 nm red light, or both, at discrete, preselected regions along a plane of light source 27. Light source 27 may be a stand-alone panel, which may consist of an array of surface mounted light emitting diodes (SMD), or an array of chip-mounted optical output devices (COB). Other functionally-similar devices may be used for light source (panel) 27. Illuminator 20 can be powered by electricity from a power source 127, such as mains power. Alternatively, power source 127 may be a battery, or other stand-alone power source. On illuminator 20, support spars 24 and 25 can be nested between and abutting support spars 23 and 26 in, for example, a lap joint abutment, to form a rigid support frame to support light source 27. Legs 21, 22 may be used to adjust the vertical height of an anti-myopia illuminator that may be resting on a work surface, such as a table or workbench. Hinges 28 and 29 may be used to affix one anti-myopia optical illuminator 20 to others of similar type to enlarge the area of diffuse lighting and to directionally point the areas of illumination provided by each of such devices 20. In an embodiment, illuminator 20 can provide indirect or diffuse illumination into human eyes, the illumination having at least 100 lux of monochromatic red wavelengths in the range of about 640 nm to about 690 nm. It is posited that the illumination can inhibit ocular growth by biostimulation to control progressive myopia leading to excessive juvenile axial elongation in juvenile eyes. Such illumination also can control age-related macular degeneration in aging adult eyes.

Pertinent to juvenile eyes, illuminator 20 simultaneously may provide greater than about 1 lux of monochromatic violet-blue wavelengths in the range of about 440 nm to about 484 nm. The violet-blue light can be used for entrainment of biological circadian time clock activation, which is not limited to the melatonin clocks and includes melanopsin activation. Entrainment of biological circadian time clock activation by this technique is also thought to inhibit excessive juvenile axial elongation in juvenile eyes. Entrainment of biological circadian time clock activation needs some time. Typically, the melanopsin receptors enabling circadian cycle entrainment are effectively stimulated after at least about 20 minutes of irradiation. In general, illuminator 20 can be configured to inhibit ocular growth, by biostimulation to control excessive juvenile axial elongation in juvenile eyes, which may lead to progressive myopia. In another embodiment, it may be desirable to provide at least 65% of the output energy of the red light, relative to the blue light. At the same time, minimization of wavelengths from about 484 nm to 640 nm, with substantial elimination of wavelengths at or near to about 550 nm can be useful in reducing undesirable pupil dilation and limiting peripheral ocular tissue exposure to light. Typically, illuminance values can be from about 2,000 lux or more to about 30,000 lux or less, giving a nominal indirect total combined light exposure of about 9000 lux.

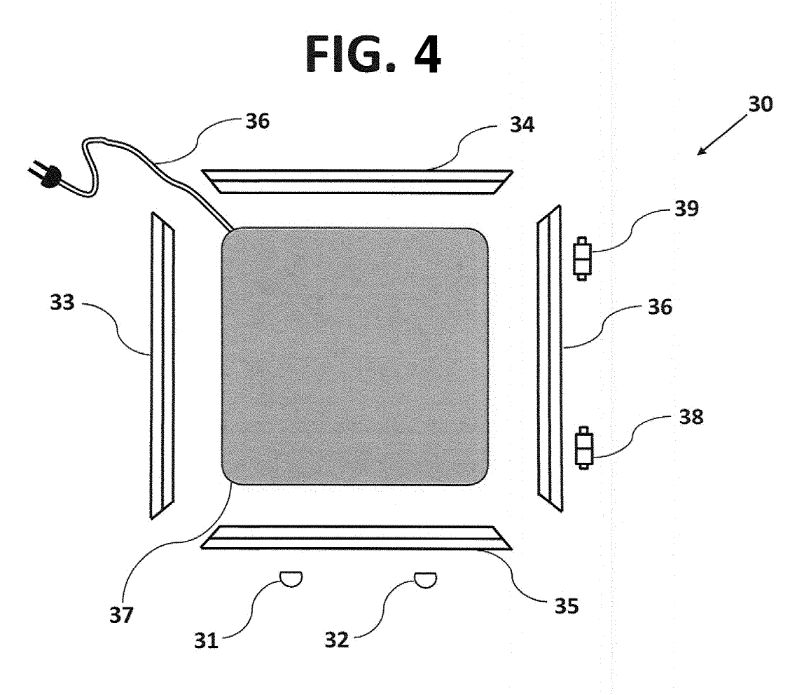

FIG. 4 depicts an alternative form of support spars 23-26 of FIG. 3, with a beveled shape. In addition, although illuminator 20 is depicted as a “panel,” other shapes may be used, especially when illuminator 20 is configured as a handheld device or a wearable ocular device. A wearable ocular device may be an ocular prosthetic device, which may be positioned or worn on or near the human face to supplement ambient or solar light with metered and controlled artificial irradiation to accumulate the daily diurnal ocular input. Also, illuminator 20 of FIGS. 3-4 is illustrated in a rectilinear form, it also can be provided in a curvilinear form.

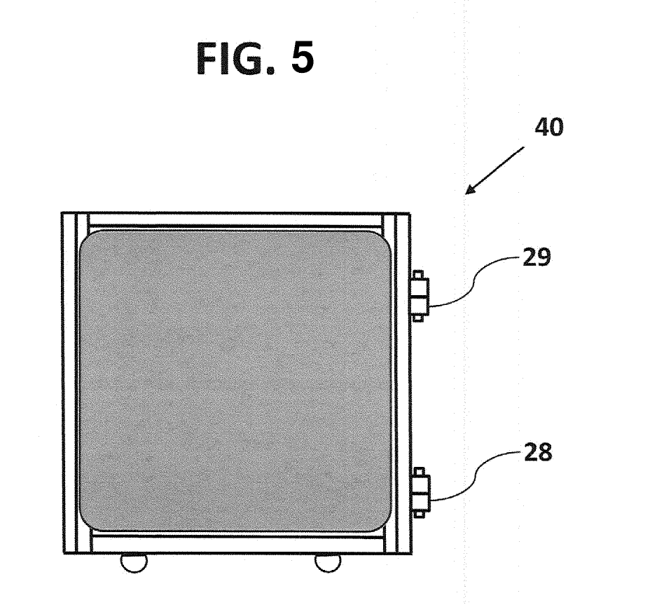

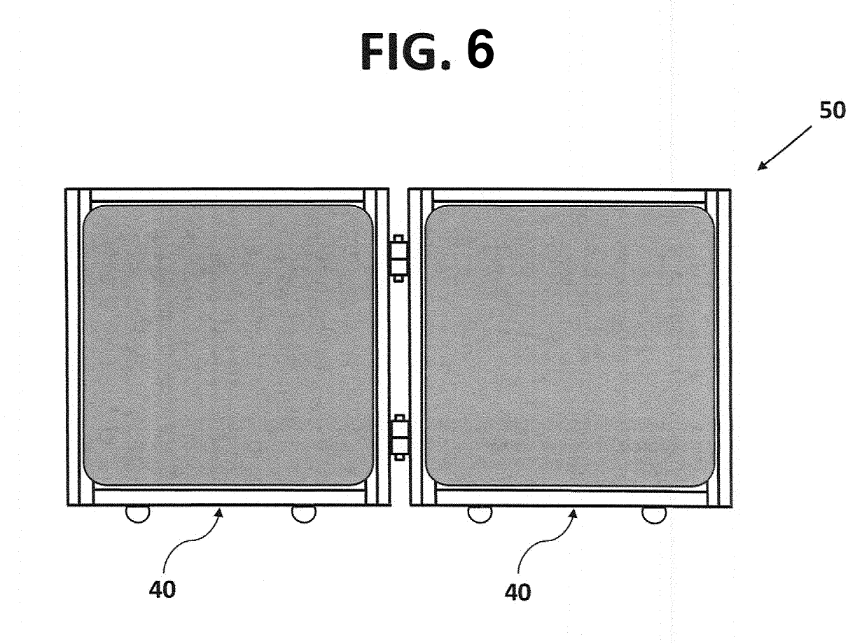

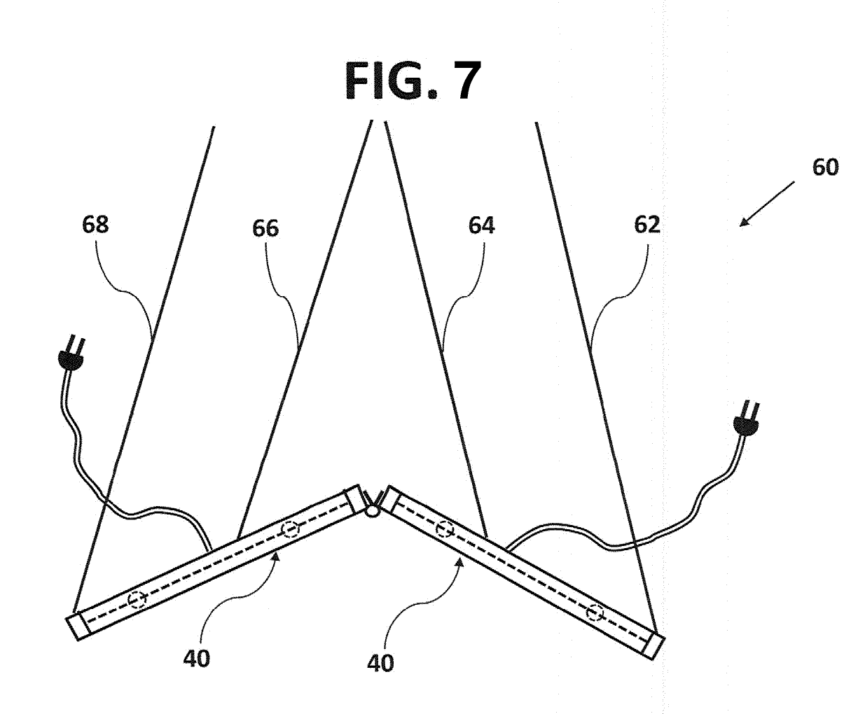

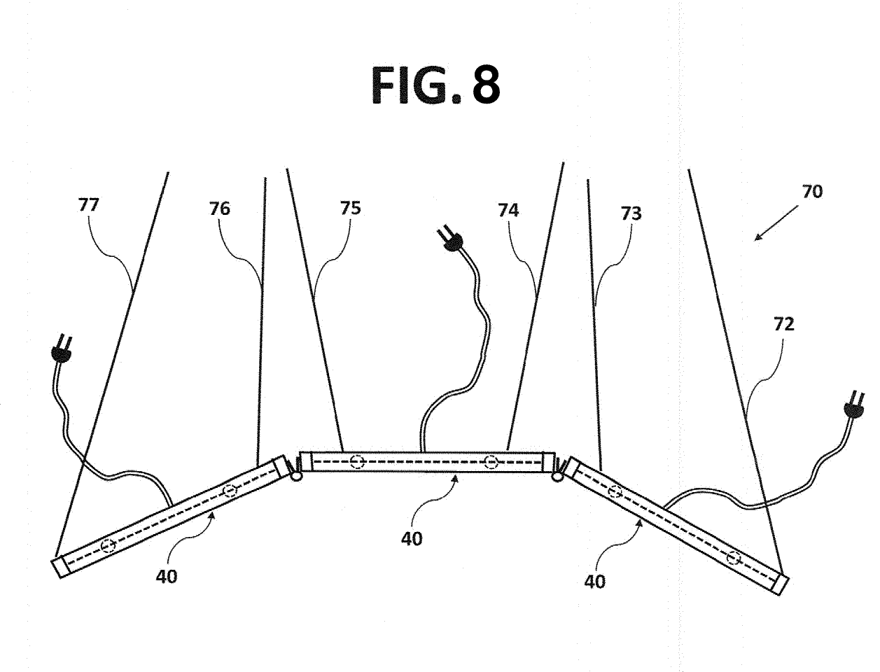

FIG. 5 depicts a plan view of a one therapeutic illuminator panel arrangement 40, and FIG. 6 depicts a plan view of a two therapeutic illuminator panel arrangement 50, which employ the support spars depicted which can be joined by hinges 28, 29. FIG. 7 depicts a top view of the two illuminator panel arrangement 50 of FIG. 6. In FIG. 7, dual illuminator arrangement 50 can be supported by hanging chains or cables 62, 64, 66, 68. Similarly, FIG. 8 depicts a top view of a three therapeutic illuminator panel arrangement 70. In the arrangement 70 of FIG. 8, illuminator panels 40, 50, and 70 can be supported by hanging chains or cables 72-77. Panels 40, 50, and 70 can be arranged to provide a selected amount of optical irradiation to selected portions of one or both eyes.

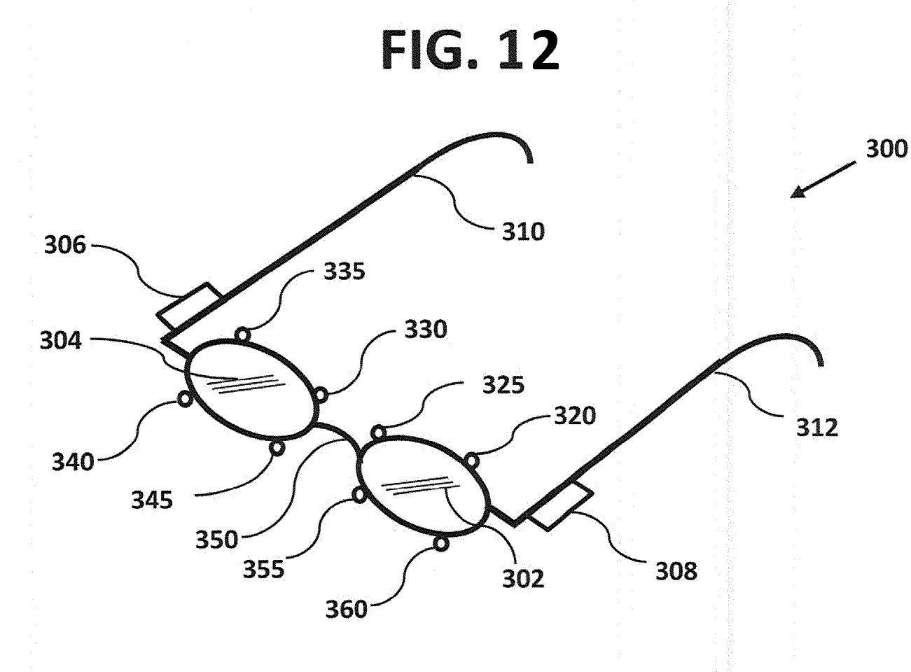

In yet another alternative embodiment, illuminator 20 can be configured to provide a system for long term delivery of irradiation for treatment of ocular tissue in connection with a digital information display. In particular, illuminator 20 may be configured to be a therapeutic handheld device, as depicted in FIGS. 8-11, or a therapeutic, wearable ocular device, such as, without limitation, an eye mask, or a pair of glasses or goggles, as depicted in FIGS. 12-14. The digital information display may be configured to simultaneously produce visible graphic images as well as invisible, diffuse non-graphic and non-display irradiation emissions. The visible graphic images and invisible, diffuse non-graphic and non-display irradiation emissions may be configured to direct into human eyes. In such embodiments, illuminator 20 can produce greater than 2 Watts per areal centimeter of invisible, continuous, diffuse non-graphic monochromatic Near Infrared (NIR) irradiative light emission directed primarily at ocular tissues.

In general, the NIR light has substantially no perceptible display color and creates no perceptible image. The NIR light may use wavelengths of about 690 nm to about 950 nm, and have a spectral full width at half maximum of less than 150 nm. Such illumination may be used in part for inhibiting ocular growth in that type of progressive myopia leading to excessive axial elongation in juvenile human eyes. Such illumination also can increase at least one of perfusion by blue light-initiated regulatory hormones, by ocular blood flow, or by ocular tissue oxygenation. This perfusion enhancement facilitates the transport of nocturnal circadian hormones to better reach substantially all ocular tissues. The presentation of the NIR invisible light can be simultaneously combined with at least about 1 Lux of ambient visible or solar light, which contains blue wavelengths of irradiance from about 400 nm to 480 nm. The NIR light and visible or solar light can be rendered by transmission, reflection, or refraction into one or both eyes. Typically, the non-visible, non-graphic output can be configured to be at least about 65% in the output energy arriving at the eye, as compared to the indirect ambient contextual light plus any quantity of the user-directed artificial graphical information display light emission. This embodiment also is considered to inhibit ocular growth by biostimulation to control progressive myopia leading to excessive juvenile axial elongation in juvenile eyes, as well as to inhibit and control age-related adult macular degeneration.

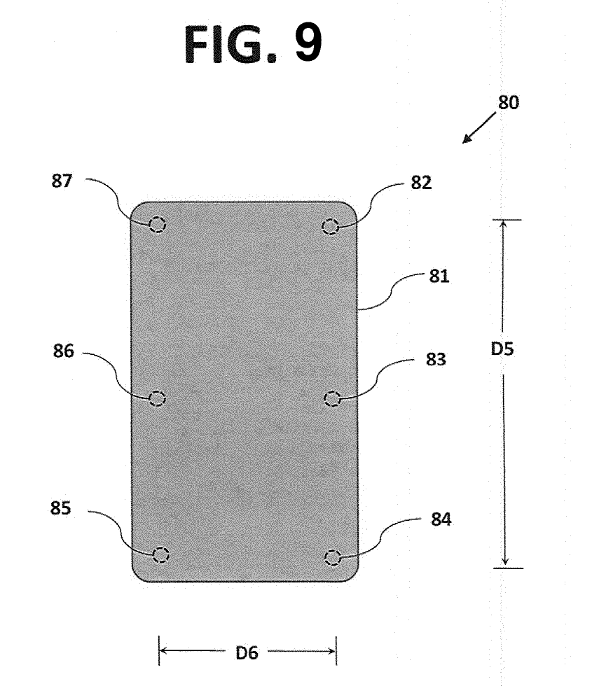

FIG. 9 illustrates a “smart phone” telecommunication and computing device (“smart phone”), having supplemental anti-myopia ocular illuminating sources 82, 84, 85, and 87. Sources 82, 84, 85, 87 may be embedded in the front (user-facing) face 81, and may be disposed on the surface or subsurface. In general, illuminating sources 82, 84, 85, 87 can emit infrared light at a nominal wavelength of about 710 nm to 860 nm, and also may emit violet light at a nominal wavelength of about 365 nm. Alternatively, light sources may include ocular illuminating sources 82, 83, 84, 85, 86, 87. Typically, emissions of the functional and interactive display of graphical information and control icons may be generated from an area bounded approximately by dimensions D5 and D6. Violet and infrared light may be distributed into the eyes of the user by this device, by direct or indirect illumination. For example, it may be desirable to reduce the number of illuminating sources 82, 83, 84, 85, 86, 87, while providing graphical displays additionally capable of producing light wavelengths with intensities equivalent to those produced by the set of ocular directed LED devices 82, 83, 84, 85, 86, 87. In all cases, the result of such a specification must be able to induce the required inhibitory physiological growth response in the eyeballs of the juvenile user.

In still another embodiment of a handheld ocular device, such as device 80 in FIG. 9, NIR-wavelength LEDs may be installed on a commercially available cell phone cell case to illuminate the eyes of the user using one or more non-visible NIR wavelength as photodynamic treatment. The LEDs can be configured to provide the function of an ocular light energy treatment device controlled by a software program, or “app” coupled to device 80. The invisible infrared LED light output dosage can be provided at wavelengths greater than about 690 nanometers, to achieve ocular growth hormone inhibition in juveniles, while the cell phone and installed phone applications are being routinely operated during ordinary use of the cell phone graphic display. The cell phone graphic display also produces visible light output during such treatment.

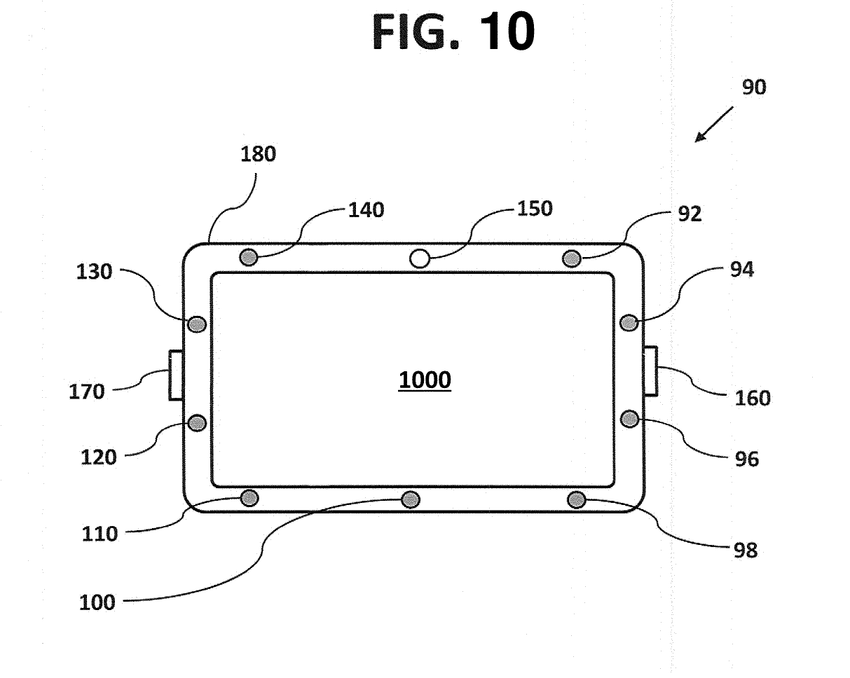

Referring now to FIG. 10, “Supplemental Ocular Light Dose Adapter” frame 180 is illustrated, which can be attached to the perimeter of a visual graphical display or an interactive graphical display region of a smart phone or a handheld tablet (1000). Frame 180 may be made from thermoplastic materials, for example, without limitation, nylon, ABS (acrylonitrile butadiene styrene), or hard rubber. Frame 180 can be secured to the display region using two or more clips 160, 170, which may be, without limitation, metallic or plastic clips. Tensioning straps made of, without limitation, plastic or woven materials, can be tensioned between clips 160, 170 such that the position of frame 180 can be securely bound to the perimeter of the interactive graphical image generator or display screen. A hole or a transparent region 150 may be provided in the material of frame 180 to allow the unobstructed view of a camera lens normally used with a smart phone or laptop computer 1000.

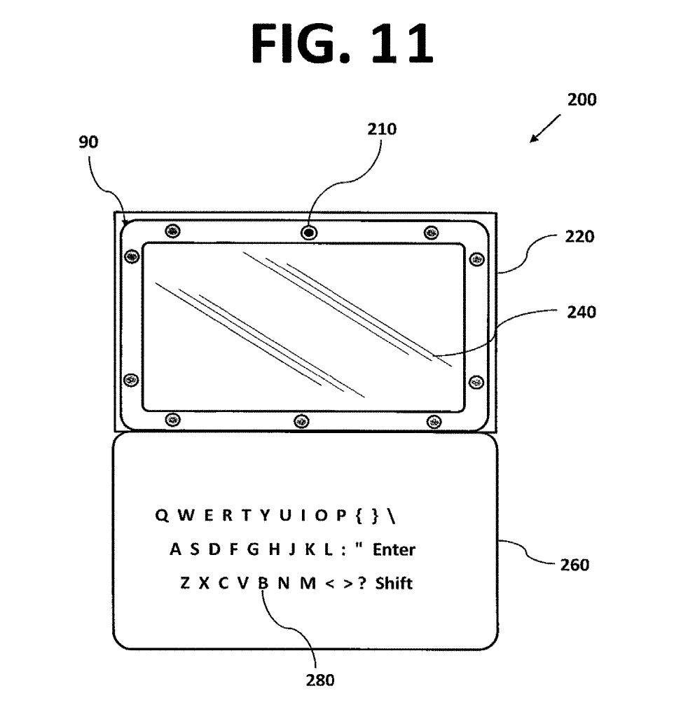

Referring now to FIG. 11, “Supplemental Ocular Light Dose Adapter” 90 can be mounted to a personal computer or laptop computer display screen 220 having a transparent screen 240 capable of passing light to generate a visual graphic display for the viewer of the screen. Device 90 can be fitted with a camera 210, and a keyboard component 260. Keyboard component 260 can be provided with independent keys or a touch surface interface having characters that are printed or projected to component 260 to accept manual user input in region 280. The supplemental ocular light dose adapter 90 can provide light having wavelengths capable of inducing an inhibitory physiological growth response in the eyeballs of the juvenile user, as described herein. Adapter 90 may be removable or may be made a permanent part of such a computer.

FIG. 12 illustrates corrective eyewear 300 having a pair of corrective lenses 302 and 304. The traditional purpose of transparent corrective lenses 302, 304 is to adjust the visual field for sharpened visual acuity. Eyewear 300 can be configured to be worn on the face, having a nose bridge 350, a right ear support 310, and a left ear support 312. Eyewear also may include a right battery for electric supply 306 and a left battery for electric supply 308. Eyewear 300 also can include light emitting diodes (LED) powered by electric supply 306, electric supply 308, or both. The LEDs can provide light having wavelengths capable of inducing an inhibitory physiological growth response in the eyeballs of the juvenile user, as described herein. LEDs can be installed at locations 320, 325, 330, 335, 340, 345, 355, and 360 such that light from these devices is significantly directed into the eyes of the wearer of the corrective lenses. Those light generating components inducing an inhibitory physiological growth response in the wearer's left eye represented by 355, 360, 320, 325 may be made independently controllable to dose that eye separately from the right eye light generating devices 330, 335, 340, 345, as a part of the function of such corrective eyewear.

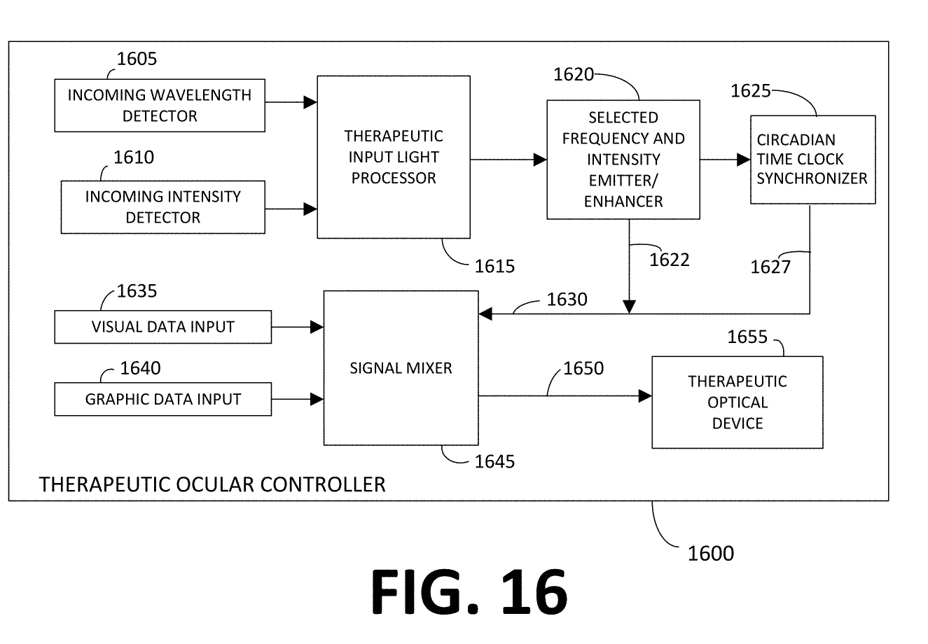

An additional function of corrective lenses 302, 304 is to self-darken the transparent glass to reduce the amount of ambient sunlight that reaches the eyes under unusually bright conditions. However, an embodiment herein, such as one including therapeutic optical controller 1600 (FIG. 16), provides a corrective supplemental and artificial light irradiation dosage metered independently to each eye of the user to selectively inhibit ocular growth to each eye as needed by the utility of corrective eyewear 300, such that refractive properties of 302, 304 may not be required.

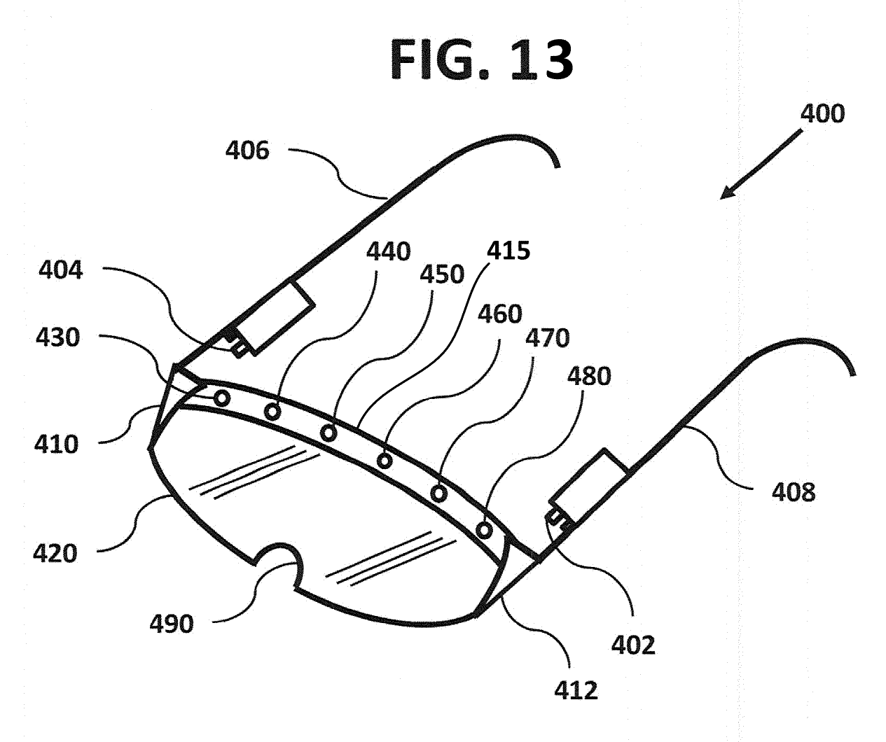

FIG. 13 illustrates a pair of goggles 400 to be worn on the face of a user for the purpose of providing a head's-up display (HUD) overlay onto the natural visual field observed through the substantially transparent screen 420. HUD goggle 400 can be configured to be worn on a human head, to send, transmit, or reflect light for omnidirectional observation by at least one eye. Goggles 400, including screen 420, are typically made from moldable thermoplastic materials such as, without limitation, polycarbonate. Screen 420 can have a partially reflective mirrored surface that is substantially transparent, and can provide nosepiece opening 490. HUD goggles 400 may include projectors 402 and 404 to interact with the viewing surface of screen 420. However, some newer types of commercially-available HUD contain an imbedded graphical display device that is substantially transparent but is otherwise capable of generating visual display information of graphical nature at 420 to be directed into the eyes of the wearer of this HUD 400 without using projector 402, 404. Earpieces 406, 408 can help to maintain positional stability of the HUD 400 on the head of the wearer by resting on the ears of the wearer. The HUD device can further be supported by upper frame 415 bounded by the distal ends of each side at 410 and 412. Upper frame 415 can be supplied with light emitting devices 430, 440, 450, 460, 470, 480 (e.g., LEDs) each of which can provide wavelengths of light directed at the eyes of the wearer that are capable of inducing an inhibitory physiological growth response in the eyeballs of the juvenile user, as described herein. Light emitting devices 430, 440, 450, 460, 470, 480 may also be configured to generate optimal frequencies of light to induce the inhibitory physiological growth response in the wearer's eyes. Such irradiation may be made independently controllable to dose each eye separately as a part of the function of such device 400. Such control may be made possible, for example, by therapeutic optical controller 1600 (FIG. 16).

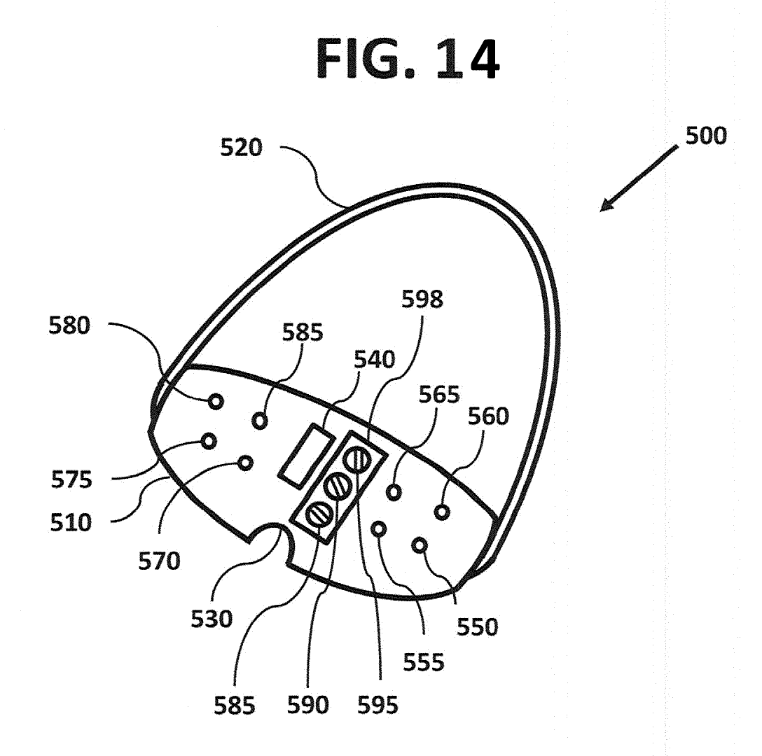

Referring now to FIG. 14, a soft ocular sleeping mask 500 is illustrated, which may be worn on the face of a child for the dual purpose of generating optimal frequencies of light at metered dosages to induce the inhibitory physiological growth response independently in each of the wearer's eyes, while substantially obstructing or filtering out the transmission of uncontrolled or variable ambient visible light from the environment that could be associated with sleep disturbances. Mask shield 510 of mask 500 may be constructed of material such as a velvet cloth or other pliable opaque material, capable of conforming to the topology and features of a human face. Typically, nose bridge 530 provides a gap or loosened area in shield 510 to allow the user's nose access to air. Securing band 520 can be a band of elastic material such as, without limitation, rubber or reversibly stretching polymer. Band 520 can wrap around the head and over the ears of a sleeping person to secure the conformable mask shield 510 onto and around the surfaces of closed eyes during sleep. Soft ocular sleeping mask 500 can be provided with an electric power source 540, which may comprise one or more batteries or rechargeable batteries capable of operating the light emission devices during lengthy durations of sleep.

LED can be provided for the left eye 550, 555, 560, 565, and for the right eye 570, 575, 580, 585, respectively. LEDs can include blue light LEDs, red light LEDs, and infrared LEDs. Blue light LEDs 560, 565, 580, 585 can have a nominal wavelength of about 460 nm. Red light LEDs 550, 575 can have a nominal wavelength of about 660 nm. Infrared LEDs 555,570 can have a nominal wavelength of about 690 nm to about 950 nm. The LEDs irradiative outputs are directed at the eyes of the wearer. Control panel 598 may be provided and may contain an electric control circuit, for example, therapeutic optical controller 1600 (FIG. 16), and control knobs to regulate the light produced by the light emitting devices, where the duration, intensity and duty cycle of these lights may be programmable and adjusted such as by variable resistor trimming potentiometers 585, 590, 595. Alternatively, a controller, such as limitation, therapeutic optical controller 1600 (FIG. 16), may be used without potentiometers 585, 590, 595.

A plurality of control functions may be provided. One function may be to shift the irradiance duty to a greater ratio or a lesser ratio of the higher to lower wavelength light emission devices using variable resistor trimming potentiometer 585. Another function may be to alter the duration of the light to a comfortable and gentle sinusoidal fade on and fade off period over hours or minutes depending on the preference of the user's need to minimize sleep disturbances by adjusting variable resistor trimming potentiometer 590. Yet another feature of ophthalmic sleeping mask 500 may be to provide a greater or lesser fixed intensity of light dosage as adjusted by variable resistor trimming potentiometer 595. Other programmable light functions are contemplated and variations can be applied to the device operation, as long as these adjustments achieve the ocular treatment objective, e.g., induction of the inhibitory physiological growth response in a juvenile wearer's eyes.

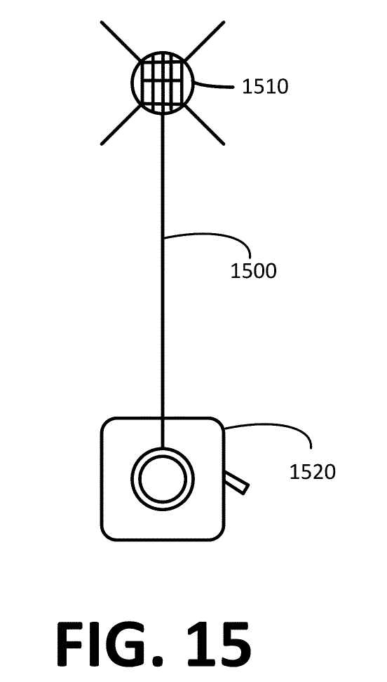

FIG. 15 depicts still another ocular device embodiment, which can be a non-wearable system for long term delivery of treatment of ocular tissue, and which can be regulated by controller 1520. The system can include lamp or lighting fixture 1500 having selected LEDs 1510. LEDs 1510 can provide indirect or diffuse illumination, and which can be directed into human eyes. The LEDs 1510 of lamp 1500 provide greater than 100 lux of monochromatic red wavelengths in the range of about 640 nm to about 690 nm light, for inhibiting ocular growth by biostimulation to control progressive myopia leading to excessive juvenile axial elongation in juvenile eyes. LEDs 1510 of lamp 1500 also can provide greater than 1 lux of monochromatic light having violet-blue wavelengths in the range of about 440 nm to 484 nm for entrainment of biological circadian time structure clock activation required for which is not limited to the melatonin clocks and includes melanopsin activation. The output energy of the red light may be at least 65% of the output energy of the blue light.

Illumination by greater than 100 lux of monochromatic red light having wavelengths in the range of about 640 nm to about 690 nm light may control macular degeneration in aging adult eyes. LEDs 1510 can be configured to limit peripheral ocular tissue exposure to light by minimizing wavelengths from about 484 nm to about 640 nm with substantial elimination of wavelengths at or near about 550 nm, which may cause undesirable pupil dilation. In another embodiment, lamp 1500 can produce greater than about 2000 Lux and less than about 30,000 Lux with nominally about 9000 lux indirect total combined light exposure. The lamp also may produce greater than about 2 W per areal centimeter of invisible, continuous, diffuse monochromatic near infrared (NIR) light having wavelengths of about 690 nm to about 950 nm, having a spectral full width at half maximum of less than about 150 nm, The light produced by the lamp may be directed at ocular tissues.

Lamp 1500 may constitute an ocular apparatus having an illuminator 1510, such as a plurality of LEDs, regulated by controller 1520. Lamp 1500 may be configured to illuminate human eyes with at least 100 lux of monochromatic light having red wavelengths in the range of about 640 nm to about 690 nm. Illuminator 1510 further may be configured to provide illuminance values from about 2,000 lux to about 30,000 lux, with a nominal indirect total combined light exposure of about 9000 lux. In embodiments, illuminator 1510 further may be configured to provide greater than about 1 lux of monochromatic light having violet-blue wavelengths in the range of about 440 nm to about 484 nm. In other embodiments of the ocular apparatus, illuminator 1510 also may be configured to minimize light having wavelengths from about 484 nm to about 640 nm, and configured to substantially eliminate light having of wavelengths at or near to about 550 nm, wherein the melanopsin receptors enabling circadian cycle entrainment are stimulated at a greater amount than that of rod and cone receptors functioning to interpret environmental visual information. In still other embodiments, illuminator may 1510 additionally be configured to provide visible display images and invisible illumination, wherein the invisible illumination comprises greater than about 2 Watts per areal centimeter of invisible, continuous, diffuse non-graphic monochromatic Near Infrared (NIR) light directed at ocular tissues.

Also contemplated is an ocular treatment system, which may include embodiments described relative to FIGS. 5-15, for long term delivery of irradiation for treatment of ocular tissue in connection with a digital information display for simultaneously producing visible display images, which may include graphic images, and invisible irradiation for direction into human eyes. In general, the invisible irradiation produced by the system can be greater than about 2 W per areal centimeter of invisible, continuous, diffuse monochromatic Near Infrared (NIR) irradiation using wavelengths of about 690 nm to about 950 nm, having a spectral full width at half maximum of less than about 150 nm. The irradiation can be directed to ocular tissues, and be used for inhibiting ocular growth, particularly excessive axial elongation in juvenile human eyes, which may lead to progressive myopia. The system also may provide simultaneous presentation of invisible light in substantially continuous combination with greater than about 1 Lux of ambient visible, or solar, light by transmission, reflection, or refraction into one or both eyes. The ambient visible, or solar light contain blue wavelengths from about 400 nm to about 480 nm.

Further, the ocular devices in FIGS. 5-15 can be used to control axial human eyeball growth in juvenile progressive myopia by providing a dosage of light over a daylight period of about 4 to about 8 hours, by stimulating growth-inhibiting neural photoreceptors including myopsin and related metamyopsin, followed by a substantial period of nocturnal darkness over a period of about 4 hours to about 8 hours to remove this inhibition. This selective illumination of one or both eyes can regulate the diurnal and nocturnal ocular tissue growth processes for respective periods of arrested growth and continued growth in accordance with a predetermined dosage, and distribution of wavelengths, of light entering the eye.

FIG. 16 illustrates therapeutic ocular controller 1600, which may be used by one or more of the therapeutic ocular devices herein. Controller 1600 can generate the duration, or maintain or adjust the preferred illumination direction of the specified ocular irradiation. Intensity and frequency of the output light also may be controlled. Controller 1600 may have incoming light wavelength detector 1605 and incoming light intensity detector 1610 coupled to therapeutic input light processor 1615. Processor 1615 can produce signals that are representative of the frequencies and intensity of received light or, alternatively, signals, which indicate a reduced value of therapeutic light in the received light. Processor 1615 signals may be received by selected frequency and intensity emitter/enhancer (SFIE) 1620. SFIE 1620 may produce signal 1622 that is indicative of which output frequencies need to be emitted or enhanced, given current control inputs (such as potentiometers 585, 590, 595 in FIG. 14). Signal 1622 also may indicate whether output light intensity needs to be increased or decreased in order to inhibit ocular growth in juveniles. Signal 1622 may be supplied directly or indirectly to therapeutic output device interface 1655, which may be coupled to a therapeutic ocular device in accordance with present embodiments. Alternatively, circadian time clock synchronizer module 1625 may be included to produce synchronization signal 1627 which may be rendered to interface 1655. Synchronization signal 1627 can produce light frequency and light intensity information, which can entrain the user's circadian rhythm and may be used to inhibit ocular growth. In embodiments which include visual or graphical data input, such as a HUD or handheld device, visual data input 1635 and graphic data input 1640 may be received by controller 1600 and transmitted to signal mixer 1645. Signal mixer can be used, for example, to produce a unified ocular display that includes therapeutic light, such as represented by signal 1622, 1627, visual data input 1635, and, if present, graphic data input 1640. Many variations of therapeutic optical controller 1600 are contemplated, including greater or lesser functionality, and discrete elements or a miniaturized integrated circuit.

In yet another embodiment, an ocular device, such as those described in FIGS. 5-15, can be configured to produce a long-term diurnal illumination dosage substantially composed of a relatively narrow band of emission wavelengths having a nominal maximum wavelength of about 730 nm peak light emission, with total combined radiant intensities not substantially greater than the energy of about 30,000 LUX or that energy equivalent to that of the ambient indirect light of day available at noon on the equator of Earth. In conjunction with the foregoing embodiments of illuminator 20 (e.g., FIGS. 5-15), riboflavin (Vitamin B2) can be administered to enhance the salutary effects of the light administration, in an amount of approximately the recommended daily allowance (RDA) of riboflavin. Riboflavin is believed to maintain cumulative diurnal photochemical cross-linking in ocular support tissues substantially other than the ocular lens tissues during periods of ocular irradiance with other than UV light. The RDA of riboflavin can be dependent on age, gender, and reproductive status.

Examples of the RDA of riboflavin dosages may be found at https://medlineplus.gov/druginfo/natural/957.html. It may be useful to take riboflavin in the morning with food. Further, nocturnal caloric intake may be minimized or eliminated. Moreover, nocturnal exposure to visible light during sleeping can be minimized or eliminated, for example, when the eyelids are closed for greater than about 2 hours, or by wearing, for example, a sleep mask. Barring nocturnal exposure to visible light can properly maintain the entrainment of the nocturnal circadian rhythm of controlled ocular growth.

A portion of the visible ambient and reflected light sources are each structured with lenses, reflective films, or the like to carry one or more visible graphic image overlays to cooperatively produce a display region immediately proximate the user's head. In another embodiment of a wearable ocular device, visual environmental information may be provided by one or more corrective non-contact refractive lenses to be worn on the head of a human being to transmit light for omnidirectional observation by at least one eye. In such embodiments, greater than about 2 Watts per areal centimeter of invisible, continuous, diffuse non-graphic monochromatic Near Infrared (NIR) irradiative light emission can be directed primarily at ocular tissues. The NIR light may use wavelengths of about 690 nm to about 950 nm, and have a spectral full width at half maximum of less than about 150 nm is directed, for example, substantially in an arc segment configuration to maximize irradiation to the upper half of the eye.

Still another embodiment provides a natural photosensitizer supplement to the diet, which photosensitizer can be in the form of natural porphyrins as organic photosensitizers having natural bioflavinoid antioxidants. The natural photosensitizer supplement can be bee propolis. The photosensitizer may be administered prior to ocular treatment or when the ocular system is being used for treatment for more than about 20 minutes.

Also provided are methods for illuminating a human eye to achieve an ophthalmic treatment. Method 1700 can include illuminating (S1705) the human eye with greater than about 100 lux of monochromatic light having red wavelengths in the range of about 640 nm to about 690 nm. Illuminating (S1705) the eye in this way may be beneficial for both a juvenile eye, as well as an aging adult eye. Other embodiments of method 1700, which may be suited for controlling progressive myopia associated excessive axial elongation in a juvenile human eye, and which may additionally include illuminating (S1710) the human eye with greater than about 1 lux of monochromatic light having violet-blue wavelengths in the range of about 440 nm to about 484 nm, illuminating (S1715) the human eye with light having illuminance values from about 2,000 lux to about 30,000 lux, and minimizing (S1720) illuminated light having wavelengths from about 484 nm to about 640 nm. In embodiments of method 1700, treatments for progressive myopia in a juvenile human eye, and macular degeneration in an adult eye also may include illuminating (S1725) the human eye with visible digital display images and invisible, diffuse irradiation, wherein the invisible, diffuse irradiation includes greater than about 2 Watts per areal centimeter of invisible, continuous, diffuse non-graphic monochromatic near-infrared light (NIR) having wavelengths from about 690 nm to about 950 nm, and having a spectral full width at half maximum of less than about 150 nm, wherein the NIR light is directed at ocular tissues, and increasing at least one of perfusion by blue light-initiated regulatory hormones, by ocular blood flow, or by ocular tissue oxygenation. This perfusion enhancement facilitates the transport of nocturnal circadian hormones to better reach substantially all ocular tissues. Method 1700 also may include illuminating (S1730) the human eye with at least about 1 Lux of ambient visible light, wherein the visible light contains blue wavelengths from about 400 nm to about 480 nm. Methods S1705 to S1730 tend to control progressive myopia leading to excessive elongation in a juvenile human eye. This control can use diurnal (daytime) blue exposure limited to no more than about 10 hours to entrain the daytime circadian hormone response. This period can be followed by at least about 7 hours where incident light, of less than about 600 nm, is substantially eliminated during nocturnal (sleeping hours) to entrain the night-time circadian hormone response.

For aging adults, selected elements of method 1700 may enhance ocular health, and treat human ophthalmic conditions by improving or maintaining blood flow to the retinal tissues, and the retinal attachment points, which may avoid, control, or substantially reduce macular degeneration in aging adults.

The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law. Moreover, it is noted that like reference numerals represent similar parts throughout the several views of the drawings, although not every figure may repeat each and every feature that has been shown in another figure in order to not obscure certain features or overwhelm the figure with repetitive indicia. It is understood that the invention is not limited to the specific methodology, devices, apparatus, materials, applications, etc., described herein, as these may vary. It is also to be understood that the terminology used herein is used for the purpose of describing particular embodiments only, and is not intended to limit the scope of the invention.

1. An illumination apparatus, comprising:
an illuminator configured to illuminate human eyes with at least 100 lux of monochromatic light having red wavelengths in the range of about 640 nm to about 690 nm, and configured to increase at least one of perfusion by blue light-initiated regulatory hormones, by ocular blood flow, or by ocular tissue oxygenation.

2. The illumination apparatus of claim 1, wherein the illuminator is configured to control progressive myopia leading to excessive axial elongation in a juvenile human eye.

3. The illumination apparatus of claim 1, wherein the illuminator is configured to control macular degeneration in an aging adult human eye.

4. The illumination apparatus of claim 1, wherein the illuminator is configured to provide indirect light or diffuse light.

5. The illumination apparatus of claim 1, wherein the illuminator is configured to provide illuminance values from about 2,000 lux to about 30,000 lux, with a nominal indirect total combined light exposure of about 9000 lux.

6. The illumination apparatus of claim 1, wherein the illuminator comprises a wearable ocular device.

7. The illumination apparatus of claim 1, wherein the illuminator comprises a handheld device.

8. The illumination apparatus of claim 1, wherein the illuminator comprises a stand-alone device.

9. The illumination apparatus of claim 2, wherein the illuminator is configured to provide greater than about 1 lux of monochromatic light having violet-blue wavelengths in the range of about 400 nm to about 484 nm.

10. The illumination apparatus of claim 2, wherein the illuminator is configured to minimize light having wavelengths from about 484 nm to about 640 nm, and configured to substantially eliminate light having of wavelengths at or near to about 550 nm, wherein the melanopsin receptors enabling circadian cycle entrainment are stimulated at a greater amount than that of rod and cone receptors functioning to interpret environmental visual information.

11. An illumination apparatus of claim 5, wherein the illuminator is further configured to provide visible display images and invisible illumination, and wherein the invisible illumination comprises greater than about 2 Watts per areal centimeter of invisible, continuous, diffuse non-graphic monochromatic Near Infrared (NIR) light directed at ocular tissues, and configured to increase at least one of perfusion by blue light-initiated regulatory hormones, by ocular blood flow, or by ocular tissue oxygenation.

12. The illumination apparatus of claim 6, wherein the wearable ocular device comprises one of an eye mask, goggles, or a pair of glasses.

13. The illumination apparatus of claim 7, wherein the handheld device comprises one of a phone, a tablet computer, or a laptop computer.

14. The illumination apparatus of claim 8, wherein the stand alone device comprises at least one illumination panel.

15. A method for illuminating a human eye, comprising:
illuminating the human eye with greater than about 100 lux of monochromatic light having red wavelengths in the range of about 640 nm to about 690 nm.

16. The method of claim 15, further comprising:
illuminating the human eye with greater than about 1 lux of monochromatic light having violet-blue wavelengths in the range of about 400 nm to about 484 nm;
providing the human eye with illuminated light having illuminance values from about 2,000 lux to about 30,000 lux; and
minimizing illuminated light having wavelengths from about 484 nm to about 640 nm.

17. The method of claim 16, further comprising:
illuminating the human eye with visible digital display images and invisible, diffuse irradiation emission, wherein the invisible, diffuse irradiation includes greater than 2 Watts per areal centimeter of invisible, continuous, diffuse non-graphic monochromatic near-infrared light (NIR) having wavelengths from about 690 nm to about 950 nm, and having a spectral full width at half maximum of less than 150 nm, wherein the NIR light is directed at ocular tissues, and increasing at least one of perfusion by blue light-initiated regulatory hormones, by ocular blood flow, or by ocular tissue oxygenation, wherein perfusion enhancement facilitates the transport of nocturnal circadian hormones to reach substantially all of the ocular tissues.

18. The method of claim 17, further comprising illuminating the human eye with at least about 1 Lux of ambient visible light, wherein the visible light contains blue wavelengths from about 400 nm to about 480 nm,
wherein progressive myopia leading to excessive axial elongation in a juvenile human eye is controlled.

19. The method of claim 15, further comprising inhibiting progressive myopia in a juvenile human eye.

20. The method of claim 15, further comprising ameliorating macular degeneration in an aging adult eye.

21. The method of claim 17, further comprising inhibiting progressive myopia in a juvenile human eye.

22. The method of claim 18, further comprising inhibiting progressive myopia in a juvenile human eye.

23. An ocular apparatus, comprising:
an illuminator configured to illuminate human eyes with at least 100 lux of monochromatic light having red wavelengths in the range of about 640 nm to about 690 nm,
wherein the illuminator is further configured to provide illuminance values from about 2,000 lux to about 30,000 lux, with a nominal indirect total combined light exposure of about 9000 lux,
wherein the illuminator is further configured to provide greater than about 1 lux of monochromatic light having violet-blue wavelengths in the range of about 400 nm to about 484 nm,
wherein the illuminator is further configured to minimize light having wavelengths from about 484 nm to about 640 nm, and configured to substantially eliminate light having of wavelengths at or near to about 550 nm, wherein the melanopsin receptors enabling circadian cycle entrainment are stimulated at a greater amount than that of rod and cone receptors functioning to interpret environmental visual information,
wherein the illuminator is further configured to provide visible display images and invisible illumination, wherein the invisible illumination comprises greater than about 2 Watts per areal centimeter of invisible, continuous, diffuse non-graphic monochromatic Near Infrared (NIR) light directed at ocular tissues, and wherein the illuminator is further configured to increase at least one of perfusion by blue light-initiated regulatory hormones, by ocular blood flow, or by ocular tissue oxygenation.

24. The ocular apparatus of claim 22, further comprising a wearable ocular device, a handheld device, or a stand-alone device.

25. The ocular apparatus of claim 23, wherein the illuminator is configured to control progressive myopia in a juvenile human eye.

26. The ocular apparatus of claim 23, wherein the illuminator is configured to control macular degeneration in an aging adult human eye.
